(19) United States
(12) Patent Application Publication
(10) Pub. No.: US 2015/0313816 A1
(43) Pub. Date: Nov. 5, 2015

(54) COMPOSITION COMPRISING A DICARBONYL COMPOUND AND AN ACID, THE PROCESS FOR STRAIGHTENING KERATIN FIBRES USING THIS COMPOSITION

(71) Applicant: L'OREAL, Paris (FR)

(72) Inventor: Nicolas DAUBRESSE, La Celle Saint Cloud (FR)

(21) Appl. No.: 14/441,366

(22) PCT Filed: Nov. 8, 2013
(86) PCT No.: PCT/EP2013/073448

§ 371 Date: May 7, 2015

(30) Foreign Application Priority Data
Nov. 9, 2012 (FR) 1260662

(51) Int. Cl. A61K 8/368; A61K 8/24; A45D 2/00; A61Q 5/06; A45D 7/06; A61K 8/365; A61K 8/19

(52) U.S. Cl. CPC A61K 8/368; A61K 8/365; A61K 8/24; A61K 8/19; A61Q 5/06; A45D 7/06; A45D 2/001; A61K 2800/5922

(57) ABSTRACT

The present invention relates to a cosmetic composition comprising: i) one or more dicarbonyl compounds corresponding to formula (I) below, and/or derivatives thereof and/or hydrates thereof and/or salts thereof: in which formula (I), R is as defined in the description, in a specific amount; ii) one or more acids different from the compound(s) i) as defined previously; and iii) one or more specific alkalinizing agents. The subject of the invention is also a process for straightening keratin fibres using ingredients i), ii) and iii) with a step of straightening by means of a straightening iron at a temperature of at least 150° C., preferably ranging from 150 to 250° C.

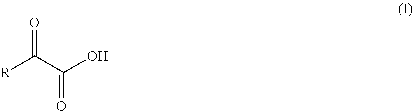

The present invention relates to a cosmetic composition, in particular hair composition, based on one or more particular dicarbonyl compounds and/or derivatives thereof and/or hydrates thereof and/or salts thereof i), and on one or more acids different from i), in particular carboxylic acids, different from said particular dicarbonyl compound(s) and also to a process for straightening the hair using this composition.

In the hair field, consumers wish to have available compositions which make it possible to introduce a temporary change to their head of hair, while targeting a good wear property for the effect produced. In general, it is desirable for the change to withstand shampooing operations for a minimum of 15 days, indeed even more, depending on the nature of said change.

Treatments already exist for modifying the colour or the shape of the hair and also, to some extent, the texture of the hair. One of the known treatments for modifying the texture of the hair consists of the combination of heat and of a formaldehyde-comprising composition. This treatment is in particular effective for conferring a better appearance on damaged hair and/or for treating long hair and curly hair.

The action of formaldehyde is associated with its ability to crosslink proteins by reaction with its nucleophilic sites. The heat used can be that of an iron (flat tongs or crimping iron), the temperature of which can generally reach 200° C. or more. However, there is an increasing desire to avoid the use of such substances, which can prove to be aggressive to the hair and other keratin materials.

Application WO2011/104282 has thus provided a novel process for semi-permanently straightening the hair which consists in applying an alpha-keto acid solution to the hair for 15 to 120 minutes, in then drying and, finally, in straightening the head of hair with an iron at a temperature of approximately 200° C. The alpha-keto acid or α-keto acid used is preferably glyoxylic acid.

However, it has been noted that the use of glyoxylic acid can result in some significant limitations; in particular, at high concentration it may not be well tolerated, in particular when the scalp is sensitive and/or irritated. Its volatility, enhanced by the use of heat (iron), can also present a problem. Furthermore, cosmetic formulations having an acidic pH can detrimentally affect the hair and/or detrimentally affect the colour thereof.

It is already known practice to use glyoxylic acid esters in hair compositions, in particular in hair dyeing compositions, as described in document DE19859722, and in reducing compositions, as described in document DE19860239.

However, the efficiency of these compounds is not yet sufficient.

The purpose of the invention is to develop a straightening/relaxing composition which is stable over time and which makes it possible to straighten/relax and/or reduce the volume of the hair in an efficient and persistent manner while limiting damage to the hair, while at the same time retaining comfort at the time of application for the user of the composition, but also for the hairdresser who applies it.

Thus, a subject of the present invention is a cosmetic composition comprising:
i) one or more dicarbonyl compounds corresponding to formula (I) below, and/or derivatives thereof and/or hydrates thereof and/or salts thereof:

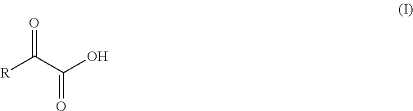

in which formula (I):
R represents an atom or group chosen from i) hydrogen, ii) carboxyl —C(O)—OH, iii) linear or branched C₁-C₆ alkyl which is optionally substituted, preferably with at least one hydroxyl —OH radical, carboxyl radical or halogen radical such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and its tautomers such as

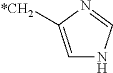

with * representing the part linked to the rest of the molecule; the dicarbonyl compounds corresponding to formula (I), and/or derivatives thereof and/or hydrates thereof and/or salts thereof being present in the composition in an amount ranging from 5 to 15% in weight of the total weight of the composition;
ii) one or more acids different from the compound(s) i) as defined previously; and
iii) one or more alkalinizing agents chosen from i) aqueous ammonia, ii) alkali metal or alkaline-earth metal carbonates or hydrogen carbonates, such as sodium carbonates or hydrogen carbonates or potassium carbonates or hydrogen carbonates, iii) alkali metal or alkaline-earth metal phosphates or (di)hydrogen phosphates, iv) alkali metal or alkaline-earth metal hydroxides, such as sodium or potassium hydroxides, or mixtures thereof, v) alkanolamines, such as monoethanolamine or trihydroxyethylamine, vi) oxyethylenated and/or oxypropylenated ethylenediamines, vii) amino acids and viii) the compounds of formula (II) below:

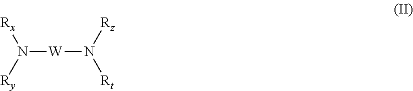

in which formula (II) W is a divalent C₁-C₆ alkylene radical optionally substituted with one or more hydroxyl groups or a C₁-C₆ alkyl radical, and/or optionally interrupted with one or more heteroatoms such as O, or NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

A subject of the invention is also a process for straightening keratin fibres, in particular the hair, using i) one or more dicarbonyl compounds corresponding to formula (I) and/or derivatives thereof and/or hydrates thereof and/or salts thereof as defined previously, the amount of dicarbonyl compounds of formula (I) and/or derivatives thereof and/or hydrates thereof and/or salts in a composition containing them ranging from 3 to 15% in weight of the total weight of the composition; ii) one or more acids different from the compound(s) i) as defined previously and, iii) one or more alkalinizing agents, it being understood that ingredients i) and ii) and iii) can be applied together or separately to the keratin fibres.

According to one particular embodiment of the invention, the composition comprising i), ii) and optionally iii) has an acidic pH, preferably greater than or equal to 1 and less than 7 and more particularly ranging from 1 to 4, better still from 1 to 3 and even better still from 1.7 to 3.

In a first particular variant of the invention, the process for straightening keratin fibres, in particular the hair, comprises the application to said fibres of the composition of the invention, followed by a step of straightening by means of a straightening iron at a temperature of at least 150° C., preferably inclusively between 150 and 250° C.

In a second particular variant of the invention, the process for straightening keratin fibres, in particular the hair, comprises the successive application to said fibres, and in any order with or without intermediate rinsing, of a composition comprising i) one or more dicarbonyl compounds corresponding to formula (I) and/or derivatives thereof and/or hydrates thereof and/or salts thereof as defined previously and of a composition comprising ii) one or more acids different from the compound(s) i) as defined previously, and iii) one or more alkalinizing agents which are present in either of the compositions or both, the amount of the dicarbonyl compounds corresponding to formula (I), and/or derivatives thereof and/or hydrates thereof and/or salts ranging from 3 to 15% in weight of the weight of the composition, the application of these compositions being followed by a step of straightening said fibres by means of a straightening iron at a temperature of at least 150° C., preferably ranging from 150 to 250° C.

In another particular variant of the invention, the process for straightening keratin fibres, in particular the hair, comprises the successive application to said fibres, and in any order with or without intermediate rinsing, of a composition comprising i) one or more dicarbonyl compounds corresponding to formula (I) and/or derivatives thereof and/or hydrates thereof and/or salts thereof as defined previously, the amount of the dicarbonyl compounds corresponding to formula (I), and/or derivatives thereof and/or hydrates thereof and/or salts ranging from 3 to 15% in weight of the weight of the composition, and of a composition comprising ii) one or more acids different from the compound(s) i) as defined previously, and of a composition comprising iii) one or more alkalinizing agents, the application of these compositions being followed by a step of straightening by means of a straightening iron at a temperature of at least 150° C., preferably ranging from 150 to 250° C.

The cosmetic composition of the invention is capable of being obtained by mixing at least 2 compositions, one comprising one or more dicarbonyl compounds corresponding to formula (I) and/or derivatives thereof and/or hydrates thereof and/or salts thereof as described hereinafter, the other comprising one or more acids different from the dicarbonyl compound(s) i) as defined previously, one or more alkalinizing agents being optionally present in either of the compositions or both.

In the present invention, the dicarbonyl compounds of formula (I) or derivatives thereof may be in free form, but also in hydrate forms thereof or in the form of salts thereof, preferably in free form or in the form of hydrates.

As “derivatives” of the dicarbonyl compounds of formula (I), mention may be made of esters of the carboxyl group(s), amides of the carboxyl group(s), and (thio)acetals and hemi(thio)acetals of the carbonyl function(s) of the compounds of formula (I), in free form or optionally in the form of salts or of hydrates, preferably in free form or in the form of hydrates.

The composition of the invention is stable. The composition of the invention, and the process for treating keratin fibres using ingredients i), ii) and optionally iii) as defined previously allow good straightening of keratin fibres while limiting damage to these keratin fibres, even when the application of the composition(s) is followed by heat treatment, in particular by means of a hair-straightening iron, and have an appreciated working quality, in particular without excessive vaporization of the composition at the time of straightening. The composition and the process for treating keratin fibres according to the invention also make it possible to limit the change in the colour of the fibres and also the problems of breaking of said fibres such as the hair. The composition and the process of the invention will also improve the physical properties of the hair, by reducing the frizziness effect in a long-lasting manner.

In that which follows, the expression “at least one” is equivalent to the expression “one or more”.

Preferably, the composition according to the invention comprises neither a colouring agent nor a reducing agent.

The term “colouring agents” is intended to mean, according to the present invention, agents for colouring keratin fibres such as direct dyes, pigments or oxidation dye precursors (bases and couplers). If they are present, their content does not exceed 0.001% by weight relative to the total weight of the composition. Indeed, at such a content, only the composition would be dyed, i.e. no dyeing effect would be observed on the keratin fibres.

It should be remembered that oxidation dye precursors, oxidation bases and couplers are colourless or only slightly coloured compounds which, by a condensation reaction in the presence of an oxidizing agent, give a coloured entity. With regard to direct dyes, these compounds are coloured and exhibit a degree of affinity for keratin fibres.

The term “reducing agent” is intended to mean, according to the present invention, an agent capable of reducing the disulfide bonds of the hair, such as the compounds chosen from thiols, alkali metal sulfites, hydrides or phosphines.

Preferably, the dicarbonyl compound(s) corresponding to formula (I) and/or derivatives thereof and/or hydrates thereof and/or salts thereof are chosen from the dicarbonyl compounds corresponding to formula (I) in which R represents i) a hydrogen atom or ii) a linear or branched C₁-C₆ alkyl group optionally substituted with a carboxyl group.

More preferably, they are chosen from glyoxylic acid and pyruvic acid, a derivative thereof and hydrates thereof or salts thereof and more preferentially from glyoxylic acid, derivatives thereof and the hydrate form thereof.

As glyoxylic acid derivatives, mention may be made of glyoxylic acid esters, glyoxylic acid amides, glyoxylic acid (thio)acetals and hemi(thio)acetals, and glyoxylic acid ester (thio)acetals and hemi(thio)acetals.

The esters and amides can be synthesized, using conventional esterification or amidation processes, from the corresponding acids well known to those skilled in the art.

Preferentially, the dicarbonyl compound(s) of formula (I) of the invention are chosen from glyoxylic acid and derivatives thereof and the hydrate forms of these compounds.

Mention may first of all be made of glyoxylic acid and also the hydrate form thereof (HO)₂CH—C(O)—OH, such as, for example, the glyoxylic acid in aqueous solution at 50% sold by the company Merck.

The glyoxylic acid esters are, for example, obtained from glyoxylic acid and a mono- or polyalcohol.

The term “mono- or polyalcohol” is intended to mean an organic compound comprising a hydroxyl group (monoalcohol) or at least two hydroxyl groups (polyalcohol or polyol), it being possible for said hydroxylated organic compound to be aliphatic, acyclic, linear or branched, or (hetero)cyclic, such as sugars (mono- or polysaccharides) or sugar alcohols.

More particularly, the polyalcohol comprises from 2 to 100 hydroxyl groups, and preferentially from 2 to 20 hydroxyl groups, even more preferentially from 2 to 10 hydroxyl groups and better still 2 or 3 hydroxyl groups.

Preferably, the mono- or polyalcohol is chosen from methanol, ethanol, propanol, isopropanol, butanol, hexanol, vethylene glycol, glycerol, dihydroxyacetone, glucose, sorbitol and menthol.

By way of esters, mention may particular be made of methyl glyoxylate, ethyl glyoxylate, glyceryl glyoxylate, dihydroxyacetone glyoxylate, glyceryl diglyoxylate or triglyoxylate, sorbitol mono-, di- or triglyoxylate, glucose mono-, di- or triglyoxylate, menthyl glyoxylate, and the acetals, hemiacetals and hydrates thereof.

The glyoxylic acid amides are, for example, obtained from glyoxylic acid and an organic mono- or polyamine.

The term “mono- or polyamine” is intended to mean an organic compound comprising an amino(monoamine) group or at least two (and preferably from 2 to 100, better still from 2 to 20) amino groups, it is possible for said organic compound to be aliphatic, acyclic, linear or branched or (hetero)cyclic.

The term “amino” group is intended to mean a primary amine group —NH₂ or a secondary amine group >NH.

Preferably, the mono- or polyamine is aliphatic.

This amine is preferably chosen from methylamine, ethylamine, propylamine, isopropylamine, butylamine, hexylamine, monoethanolamine, monopropanolamine, propane-1,2,3-triamine and diaminoacetone.

Mention may in particular be made of glyoxylic acid N-beta-hydroxyethylamide and glyoxylic acid N-gamma-hydroxypropylamide and the acetals, hemiacetals and hydrates thereof.

The glyoxylic acid (thio)acetals and hemi(thio)acetals may, for example, be obtained from the reaction of alcohols, for the acetals or hemiacetals, or of thiols, for the thioacetals or hemithioacetals, with blocked forms of glyoxylic acid, followed by hydrolysis. The alcohols can be the same as those mentioned for the esters. The thiols may be equivalents (referred to as mono- or polythiols) to the mono- or polyalcohols mentioned above, except for the fact that the hydroxyl function(s) of said mono- or polyalcohols is (are) replaced with one or more thiol functions SH of the mono- or polythiols. The acetals or thioacetals can also be cyclic (thio)acetals.

Mention may in particular be made of dimethoxyacetic acid, diethoxyacetic acid, 1,3-dioxane-2-carboxylic acid and 1,3-dioxolane-2-carboxylic acid.

The salts may be salts derived from the interaction of the compounds of formula (I) with acids or bases, it being possible for the acids or bases to be of organic or inorganic nature.

Preferably, the salts are salts derived from the interaction of the compounds of formula (I) with bases. Mention will in particular be made of the salts of alkali metals or alkaline-earth metals and in particular the sodium salts.

According to one embodiment, the composition of the invention comprises from 5% to 15% of one or more dicarbonyl compounds corresponding to formula (I) and/or of a derivative thereof and/or of hydrate forms thereof and/or salts thereof, preferably from 5% to 10% by weight of the total weight of the composition.

The composition according to the invention also comprises ii) one or more acids, the acid(s) being different from the compound(s) i) as defined previously.

For the purpose of the invention, the term “acid” is intended to mean an acidifying agent capable, via its presence at 1% by weight, of reducing, at 25° C., the pH of pure water or of an aqueous-ethanolic solution containing 30% by weight of ethanol by at least 0.01 unit.

According to one particular embodiment of the invention, the acid(s) ii) of the invention is (are) chosen from the following organic or inorganic acids or mixtures thereof:
hydrochloric acid, sulfuric acid, phosphoric acid;
sulfonic acids Rₐ—S(O)₂—OH, phosphonic acids Rₐ—P(O)(OH)₂, with Rₐ representing an optionally substituted C₁-C₈ alkyl, optionally substituted (hetero)aryl or optionally substituted (hetero)aryl(C₁-C₈)alkyl group;
aromatic or non-aromatic carboxylic acids comprising at least one carboxyl function —C(O)—OH chosen from:
   C₂-C₈ monoacids corresponding to the formula R_{b}—C(O)—OH in which the R_{b} radical represents a C₂-C₈ alkyl, (hetero)aryl or (hetero)aryl(C₂-C₈)alkyl group, the alkyl part being linear or branched, the alkyl and/or (hetero)aryl part being optionally substituted, preferably with one or more hydroxyl groups, one of the hydroxyl groups preferably being separated from the carboxyl function —C(O)—OH by one or two carbon atoms;
   among these carboxylic acids, mention will preferentially be made of glycolic acid, lactic acid, benzoic acid and salicylic acid;
   C₂-C₃₀ diacids corresponding to the formula HO—C(O)—R_{c}—C(O)—OH in which the R_{c} radical represents:
      a) a single covalent bond σ;
      b) a saturated or unsaturated, acyclic, linear or branched, divalent C₁-C₂₈, in particular C₁-C₁₀, hydrocarbon-based group, which is optionally substituted, preferably with one or more hydroxyl groups, the divalent hydrocarbon-based group preferably being a C₁-C₈ alkylene group which is optionally substituted with one or more hydroxyl groups or a (C₂-C₆)alkenylene group which is optionally substituted with one or more hydroxyl groups;
      c) a (hetero)arylene group which is optionally substituted, preferably with one or more hydroxyl groups and which is preferably an arylene group such as phenylene;
      d) a (hetero)cycloalkylene group which is optionally substituted, preferably with one or more hydroxyl groups and which is preferably a cycloalkylene group such as cyclohexylene;
      e) or a divalent group resulting from the association of radicals derived from the groups defined in b), c) and/or d), such as: -(hetero)aryl(C₁-C₁₀)alkyl-; —(C₁-C₁₀)alkyl(hetero)aryl(C₁-C₁₀)alkyl-; -(hetero)aryl(C₁-C₁₀)alkyl(hetero)aryl-; or -(hetero)cycloalkyl(C₁-C₁₀)alkyl-; and more preferentially -aryl(C₁-C₆)alkyl- such as -phenyl(C₁-C₆)alkyl-;
      particularly, the diacids are chosen from those in which R_{c} represents a), b) or c); mention will more particularly be made of oxalic acid, malonic acid, hydroxymalonic acid, succinic acid, malic acid, tartaric acid, maleic acid, fumaric acid, itaconic acid, glutaric acid, adipic acid, azelaic acid and sebacic acid, phthalic acid, isophthalic acid and terephthalic acid;
   polyacids corresponding to the formula R_{d}[C(O)—OH]ₓ with x representing an integer greater than or equal to 3, preferably x ranging from 3 to 6, more particularly from 3 to 4 and in particular such that x is equal to 3; and R_{d} represents a polyvalent group chosen from:
      1) a saturated or unsaturated, acyclic, linear or branched, polyvalent C₁-C₂₈, in particular C₂-C₂₀, hydrocarbon-based group, which is optionally substituted with one or more groups, preferably hydroxyl groups, the hydrocarbon-based group preferably being a trivalent C₂-C₈ group which is optionally substituted with one or more hydroxyl groups;
      2) a polyvalent (hetero)aryl group which is optionally substituted, preferably with one or more hydroxyl groups, which is preferably an at least trivalent aryl group such as phenyl;
      3) a polyvalent (hetero)cycloalkyl group which is optionally substituted, preferably with one or more hydroxyl groups, which is preferably a cycloalkyl group such as cyclohexyl;
      4) or a polyvalent group resulting from the association of radicals derived from the groups defined in 1), 2) and/or 3), such as: (hetero)aryl(C₁-C₁₀)alkyl; (C₁-C₁₀)alkyl(hetero)aryl(C₁-C₁₀)alkyl; (hetero)aryl(C₁-C₁₀)alkyl(hetero)aryl; or (hetero)cycloalkyl(C₁-C₁₀)alkyl; and more preferentially aryl(C₁-C₆)alkyl such as phenyl(C₁-C₆)alkyl;
more particularly, the polyacids being chosen from the triacids derived from groups defined in 1), in particular of C₅-C₂₀, among which mention may be made of citric acid;
aromatic or non-aromatic sulfocarboxylic acids comprising at least one carboxyl function —C(O)—OH and at least one sulfonic function —S(O)₂—OH, such as [HO—C(O)]_{y}—R_{d}—[S(O)₂—OH]_{z} with R_{d} as defined previously for the polyacids; y and z being integers greater than or equal to 1, the sum y+z preferably being greater than or equal to 2 such as equal to 3;
the sulfocarboxylic acids preferably being of C₂-C₁₀, and the sulfonic acid group being separated from the carboxylic acid group(s) by a polyvalent (C₁-C₆)alkyl or aryl(C₁-C₆)alkyl chain, the alkyl part of which is linear or branched, optionally substituted with a hydroxyl group. Mention may in particular be made of sulfosuccinic acid, para-sulfobenzoic acid and 4-sulfosalicylic acid;
aromatic or non-aromatic phosphocarboxylic acids comprising at least one carboxyl function —C(O)—OH and at least one phosphonic function —P(O)(OH)₂, such as [HO—C(O)]_{y}—R_{d}—[P(O)(OH)₂]Z with R_{d} as defined previously for the polyacids; y and z being integers greater than or equal to 1, the sum y+z preferably being greater than or equal to 2 such as equal to 3; the phosphocarboxylic acids preferably being of C₂-C₁₀, and the phosphonic acid group being separated from the carboxylic acid group(s) by a polyvalent (C₁-C₆)alkyl or aryl(C₁-C₆)alkyl group, the alkyl part of which is linear or branched and optionally substituted with a hydroxyl group. Mention may in particular be made of phosphoglycolic acid.

Unless otherwise mentioned previously:

*When the “(hetero)aryl” radicals or the “(hetero)aryl” part of a radical are optionally substituted, said radicals may then be substituted on a carbon atom, with an atom or group chosen from: i) C₁-C₁₆, preferably C₁-C₈ alkyl, optionally substituted with one or more radicals chosen from the following radicals: hydroxyl, C₁-C₂ alkoxy, (poly)-hydroxy(C₂-C₄)alkoxy, acylamino, amino substituted with two identical or different C₁-C₄ alkyl radicals, optionally bearing at least one hydroxyl group, or the two radicals possibly forming, with the nitrogen atom to which they are attached, a saturated or unsaturated, optionally substituted heterocycle comprising from 5 to 7 ring members, preferably 5 or 6 ring members, optionally comprising another heteroatom which is identical to or different from nitrogen; ii) halogen; iii) hydroxyl; iv) C₁-C₂ alkoxy; v) (poly)hydroxy(C₂-C₄)alkoxy; vi) amino; vii) 5- or 6-membered heterocycloalkyl; viii) optionally cationic 5- or 6-membered heteroaryl, preferentially imidazolium, and optionally substituted with a (C₁-C₄) alkyl radical, preferentially methyl; ix) amino substituted with one or two identical or different C₁-C₆ alkyl radicals optionally bearing at least one hydroxyl group, amino optionally substituted with one or two optionally substituted C₁-C₃ alkyl radicals; x) acylamino (—NR—C(O)—R′) in which the R radical is a hydrogen atom, or a C₁-C₄ alkyl radical optionally bearing at least one hydroxyl group and the R′ radical is a C₁-C₂ alkyl radical; xi) carbamoyl ((R)₂N—C(O)—) in which the R radicals, which may be identical or different, represent a hydrogen atom, or a C₁-C₄ alkyl radical optionally bearing at least one hydroxyl group; xii) alkylsulfonylamino (R′—S(O)₂—N(R)—) in which the R radical represents a hydrogen atom, or a C₁-C₄ alkyl radical optionally bearing at least one hydroxyl group and the R′ radical represents a C₁-C₄ alkyl radical, a phenyl radical; xiii) aminosulfonyl ((R)₂N—S(O)₂—) in which the R radicals, which may be identical or different, represent a hydrogen atom, or a C₁-C₄ alkyl radical optionally bearing at least one hydroxyl group; xiv) carboxyl in acid or salified form (preferably salified with an alkali metal or an ammonium, which may or may not be substituted); xv) cyano; xvi) nitro or nitroso; xvii) polyhaloalkyl, preferentially trifluoromethyl;

the “(hetero)cyclic” or “(hetero)cycloakyl” radicals, when they are optionally substituted, may be substituted with at least one atom or group chosen from: i) hydroxyl; ii) C₁-C₄ alkoxy, (poly)hydroxy(C₂-C₄)alkoxy; iii) C₁-C₄ alkyl; iv) alkylcarbonylamino (R—C(O)—N(R′)—) in which the R′ radical is a hydrogen atom, or a C₁-C₄ alkyl radical optionally bearing at least one hydroxyl group and the R radical is a C₁-C₂ alkyl radical or amino optionally substituted with one or two identical or different C₁-C₄ alkyl groups which are themselves optionally bearing at least one hydroxyl group, it been possible for said alkyl radicals to form, with the nitrogen atom to which they are attached, a saturated or unsaturated, optionally substituted heterocycle comprising from 5 to 7 ring members, optionally comprising at least one other heteroatom identical to or different from nitrogen; v) alkylcarbonyloxy (R—C(O)—O—) in which the R radical is a C₁-C₄ alkyl radical or an amino group optionally substituted with one or two identical or different C₁-C₄ alkyl groups which are themselves optionally bearing at least one hydroxyl group, it being possible for said alkyl radicals to form, with the nitrogen atom to which they are attached, a saturated or unsaturated, optionally substituted heterocycle comprising from 5 to 7 ring members, optionally comprising at least one other heteroatom identical to or different from nitrogen; vi) alkoxycarbonyl (R-G-C(O)—) in which the R radical is a C₁-C₄ alkoxy radical, G is an oxygen atom, or an amino group optionally substituted with a C₁-C₄ alkyl group which is itself optionally bearing at least one hydroxyl group, it being possible for said alkyl radical to form, with the nitrogen atom to which it is attached, a saturated or unsaturated, optionally substituted heterocycle comprising from 5 to 7 ring members, optionally comprising at least one other heteroatom identical to or different from nitrogen;
the “(hetero)cyclic” or “(hetero)cycloalkyl” radicals, or a non-aromatic part of a (hetero)aryl radical, when they are optionally substituted, may also be substituted with one or more oxo groups;
a hydrocarbon-based chain is “unsaturated” when it comprises one or more double bonds and/or one or more triple bonds;
an “aryl” radical represents a fused or non-fused, monocyclic or polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
a “heteroaryl radical” represents an optionally cationic, fused or non-fused, monocyclic or polycyclic group comprising from 5 to 22 ring members, from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthylyl and the ammonium salt thereof;
a “heterocyclic radical” or “heterocycloalkyl radical” is a fused or non-fused, monocyclic or polycyclic radical comprising from 5 to 22 ring members, comprising from 1 to 6 heteroatoms chosen from a nitrogen, oxygen, sulfur and selenium atom, which can contain one or two unsaturations but is non-aromatic, such as morpholino, pyperidino, pyperazino, tetrahydrofuryl or pyrrolidyl;
a “cycloalkyl radical” is a fused or non-fused, monocyclic or polycyclic hydrocarbon-based radical comprising from 5 to 22 ring members, which can contain one or two unsaturations but is non-aromatic, such as cyclohexyl or cyclobutyl;
an “alkyl radical” is a linear or branched C₁-C₂₀, preferably C₁-C₈, hydrocarbon-based radical, such as methyl or ethyl;
an “alkenylene radical” is an unsaturated hydrocarbon-based divalent radical as defined previously, which can contain from 1 to 4 conjugated or unconjugated double bonds —C═C—; or —C(═CH₂)—; the alkenylene group particularly contains 1 or 2 unsaturation(s);
the expression “optionally substituted” assigned to the “alkyl”, “alkylene” or “alkenylene” radical or to “a hydrocarbon-based chain” implies that said radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a heterocycle comprising from 5 to 7 ring members, optionally comprising another heteroatom identical to or different from nitrogen; v) or a quaternary ammonium group —N⁺R′R″R′″, M⁻ for which R′, R″ and R′″, which may be identical or different, represent a hydrogen atom or a C₁-C₄ alkyl group, or else —N+R′R″R′″ forms a heteroaryl such as imidazolium optionally substituted with a C₁-C₄ alkyl group, and M⁻ represents the counterion of the corresponding organic acid, inorganic acid or halide;
an “alkoxy radical” is an alkyl-oxy radical for which the alkyl radical is a linear or branched C₁-C₆ and preferentially C₁-C₈ hydrocarbon-based radical;
when the alkoxy group is optionally substituted, this implies that the alkyl group is optionally substituted as defined above.

According to one advantageous embodiment of the invention, the acid(s) ii) of the invention which is (are) different from the compounds as defined previously is (are) chosen from organic acids and more particularly from aromatic or non-aromatic carboxylic acids comprising at least one carboxyl function —C(O)—OH. Preferentially, the organic acids are monocarboxylic acids, in particular chosen from glycolic acid, lactic acid and benzoic acid.

According to another particular embodiment of the invention, the acid(s) ii) of the invention is (are) chosen from inorganic acids such as phosphoric acid.

According to one particular embodiment, the composition of the invention comprises a minimal content of acids ii) which are different from the compounds as defined previously greater than or equal to 1%. Preferably, the amount of acid ii) is greater than or equal to 2% by weight relative to the total weight of the composition. Even more preferentially, the content of acid(s) ii) which is (are) different from the dicarbonyl derivatives as defined previously ranges from 2% to 10% by weight relative to the total weight of the composition.

The composition according to the invention comprises also iii) one or more alkalinizing agents.

The alkalinizing agent(s) may be inorganic, organic or hybrid.

The inorganic alkalinizing agent(s) is (are) preferably chosen from aqueous ammonia, alkali metal or alkaline-earth metals carbonates or hydrogen carbonates, such as sodium carbonates or hydrogen carbonates or potassium carbonates or hydrogen carbonates, alkali metal or alkaline-earth metal phosphates or (di)hydrogen phosphates, alkali metal or alkaline-earth metal hydroxides, such as sodium, potassium, calcium or magnesium hydroxides, or mixtures thereof.

The organic alkalinizing agent(s) are preferably chosen from organic amines with a pK_{b} at 25° C. of less than 12, preferably less than 10 and even more advantageously less than 6. It should be noted that it is the pK_{b} corresponding to the function of highest basicity. In addition, the organic amines do not comprise an alkyl or alkenyl fatty chain comprising more than ten carbon atoms.

The organic alkalinizing agent(s) is (are) chosen, for example, from alkanolamines, oxyethylenated and/or oxypropylenated ethylenediamines, amino acids and the compounds of formula (II) below:

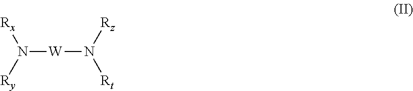

in which formula (II) W is a divalent C₁-C₆ alkylene radical optionally substituted with one or more hydroxyl groups or a C₁-C₆ alkyl radical, and/or optionally interrupted with one or more heteroatoms such as O, or NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (II) that may be mentioned include 1,3-diaminopropane, 1,3-diamino-2-propanol, spermine and spermidine.

The term “alkanolamine” is intended to mean an organic amine comprising a primary, secondary or tertiary amine function, and one or more linear or branched C₁-C₈ alkyl groups bearing one or more hydroxyl radicals.

The organic amines chosen from alkanolamines such as monoalkanolamines, dialkanolamines or trialkanolamines, comprising one to three identical or different C₁-C₄ hydroxyalkyl radicals are in particular suitable for carrying out the invention.

Among compounds of this type, mention may be made of monoethanolamine (MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in their L, D or racemic form, and comprise at least one acid function chosen more particularly from carboxylic acid, sulfonic acid, phosphonic acid or phosphoric acid functions. The amino acids may be in neutral or ionic form.

Advantageously, the amino acids are basic amino acids comprising an additional amine function optionally included in a ring or in a ureido function.

Such basic amino acids are preferably chosen from those corresponding to formula (III) below:

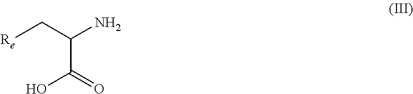

in which formula (III) Rₑ represents a group chosen from:

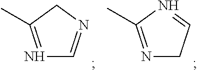

—(CH₂)₃NH₂; —(CH₂)₂NH₂; —(CH₂)₂N(H)—C(O)—NH₂; and —(CH₂)₂N(H)—C(NH)—NH₂.

The compounds corresponding to formula (III) are histidine, lysine, arginine, ornithine or citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine can also be chosen from amino acid dipeptides. As amino acid dipeptides that may be used in the present invention, mention may be made in particular of carnosine, anserine and balenine.

The organic amine can also be chosen from compounds comprising a guanidine function. Mention may in particular be made, as amines of this type which can be used in the present invention, in addition to arginine that has already been mentioned as an amino acid, of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

Preferably, the alkalinizing agent present in the composition of the invention is an alkanolamine. Even more preferentially, the alkalinizing agent is monoethanolamine (MEA).

Mention may be made, as hybrid compounds, of the salts of the abovementioned amines with acids, such as carbonic acid or hydrochloric acid.

Use may in particular be made of guanidine carbonate or monoethanolamine hydrochloride.

The alkalinizing agents according to the invention are preferably chosen from:
alkali metal or alkaline-earth metal hydroxides, such as sodium hydroxide, potassium hydroxide, slaked lime or magnesium hydroxide;
aqueous ammonia;
alkali metal or alkaline-earth metal phosphates, hydrogen phosphates or dihydrogen phosphates;
alkali metal or alkaline-earth metal carbonates or hydrogen carbonates, such as sodium hydrogen carbonate or potassium hydrogen carbonate; and
amines, alkylamines such as hydroxyalkylamines and quite particularly monoethanolamine (MEA), or triethanolamine.

The content of alkalinizing agent(s) optionally present is sufficient for the compositions to be at a pH preferably greater than or equal to 1 and less than 7, more particularly ranging from 1 to 4, better still from 1 to 3 and even better still from 1.7 to 3. Preferably, the alkalinizing agents introduced are present in a content ranging from 0.1% to 5% by weight of the total weight of the composition, better still from 0.3% to 1.5% by weight of the total weight of the composition.

The composition of the invention may also comprise at least one surfactant.

The surfactant(s) may be chosen from non-ionic, anionic, cationic, amphoteric or zwitterionic surfactants.

According to a particular embodiment, the composition comprises at least one amphoteric or zwitterionic surfactant.

In particular, the amphoteric or zwitterionic surfactant(s), which are preferably non-silicone, which may be used in the present invention may in particular be derivatives of optionally quaternized aliphatic secondary or tertiary amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines. Among the optionally quaternized secondary or tertiary aliphatic amine derivatives that may be used, as defined above, mention may also be made of the compounds of respective structures (B1) and (B2) below:

Rₐ—C(O)—N(H)—CH₂—CH₂—N⁺(R_{b})(R_{c})—CH₂—C(O)O⁻,M⁺,X⁻ (B1)

in which formula (B1):
Rₐ represents a C₁₀-C₃₀ alkyl or alkenyl group derived from an acid Rₐ—C(O)—OH preferably present in hydrolysed coconut oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine, and
X⁻ represents an organic or inorganic anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

R_{a′}—C(O)—N(H)—CH₂—CH₂—N(B)(B′) (B2)

in which formula (B2):
B represents the group —CH₂—CH₂—O—X′;
B′ represents the group —(CH₂)_{z}Y′, with z=1 or 2;
X′ represents the group —CH₂—C(O)OH, —CH₂—C(O)OZ′, —CH₂—CH₂—C(O)OH, —CH₂—CH₂—C(O)OZ′, or a hydrogen atom;
Y′ represents the group —C(O)OH, —C(O)OZ′, —CH₂—CH(OH)—SO₃H or the group —CH₂—CH(OH)—SO₃—Z′;
Z′ represents a cationic counterion derived from an alkali metal or alkaline-earth metal, such as sodium, an ammonium ion or an ion derived from an organic amine;
R_{a′} represents a C₁₀-C₃₀ alkyl or alkenyl group of an acid R_{a′}—C(O)OH preferably present in hydrolysed linseed oil or coconut oil, an alkyl group, in particular of C₁₇ and its iso form, or an unsaturated C₁₇ group.

The compounds of this type are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (B′2):

R_{a″}—N(H)—CH(Y″)—(CH₂)ₙ—C(O)—N(H)—(CH₂)_{n′}—N(R_{d})(Rₑ) (B′2)

in which formula (B′2):
Y″ represents the group —C(O)OH, —C(O)OZ″, —CH₂—CH(OH)—SO₃H or the group-CH₂—CH(OH)—SO₃—Z″;
R_{d} et Rₑ represent, independently of each other, a C₁-C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀-C₃₀ alkyl or alkenyl group of an acid R_{a′}—C(O)OH preferably present in hydrolysed linseed oil or coconut oil; and
n and n′ denote, independently of each other, an integer ranging from 1 to 3.
Among the compounds of formula (B′2), mention may be made of the compound classified in the CTFA dictionary under the name sodium diethylaminopropyl cocoaspartamide and sold by the company Chimex under the name Chimexane HB.

In accordance with a particular embodiment of the invention, the content of amphoteric or zwitterionic surfactant(s), when it (they) is (are) present, ranges from 0.05% to 30% by weight, preferably from 0.5% to 10% by weight and more preferably from 0.1% to 5% by weight, relative to the total weight of the composition.

The composition according to the invention may also comprise a cellulose-based polymer, The term “cellulose-based” polymer is intended to mean, according to the invention, any polysaccharide compound having in its structure sequences of glucose residues linked together via β-1,4 bonds; in addition to unsubstituted celluloses, the cellulose derivatives may be anionic, cationic, amphoteric or non-ionic. Thus, the cellulose-based polymers of the invention can be chosen from unsubstituted celluloses, including in a microcrystalline form, and cellulose ethers. Among these cellulose-based polymers, cellulose ethers, cellulose esters and cellulose ester ethers are distinguished. Among the cellulose esters are inorganic cellulose esters (cellulose nitrates, sulfates, phosphates, etc.), organic cellulose esters (cellulose monoacetates, triacetates, amidopropionates, acetate butyrates, acetate propionates and acetate trimellitates, etc.), and mixed organic/inorganic cellulose esters, such as cellulose acetate butyrate sulfates and cellulose acetate propionate sulfates. Among the cellulose ester ethers, mention may be made of hydroxypropylmethylcellulose phthalates and ethylcellulose sulfates.

The compositions according to the invention can be provided in any formulation form conventionally used and in particular in the form of an aqueous, alcoholic or aqueous/alcoholic solution or suspension or oily solution or suspension; of a solution or a dispersion of the lotion or serum type; of an emulsion, in particular having a liquid or semi-liquid consistency, of the O/W, W/O or multiple type; of a suspension or emulsion having a soft consistency of (O/W) or (W/O) cream type; of an aqueous or anhydrous gel, or of any other cosmetic form.

These compositions can be packaged in pump-action sprays or in aerosol containers, in order to provide for application of the composition in the vaporized (lacquer) form or in the form of a mousse. Such packaging forms are indicated, for example, when it is desired to obtain a spray or a mousse, for the treatment of the hair. In these cases, the composition preferably comprises at least one propellant.

The compositions of the invention can be aqueous or anhydrous. They are preferably aqueous and then comprise water at a concentration ranging from 5% to 98%, better still from 5% to 90% and even better still from 10% to 90% by weight relative to the total weight of the composition.

The composition may in particular comprise one or more organic solvents, in particular water-soluble solvents, such as C₁-C₇ alcohols; mention may in particular be made of C₁-C₇ aliphatic monoalcohols or C₆-C₇ aromatic monoalcohols C₃-C₇ polyols and C₃-C₇ polyol ethers, which may be employed alone or as a mixture with water.

The composition of the invention may also comprise at least one common cosmetic ingredient, chosen in particular from propellants; oils; solid fatty substances and in particular C₈-C₄₀ esters, C₈-C₄₀ acids; C₈-C₄₀ alcohols; sunscreens; moisturizers; antidandruff agents; antioxidants; chelating agents; nacreous agents and opacifiers; plasticizers or coalescers; fillers; silicones and in particular polydimethylsiloxanes; polymeric or non-polymeric thickeners or gelling agents other than the cellulose-based polymers already mentioned; emulsifiers; polymers, in particular conditioning or styling polymers other than those described previously; fragrances; silanes; crosslinking agents. The composition can, of course, comprise several cosmetic ingredients appearing in the above list.

Depending on their nature and the purpose of the composition, the normal cosmetic ingredients can be present in normal amounts which can be easily determined by those skilled in the art and which can be, for each ingredient, between 0.01% and 80% by weight. Those skilled in the art will take care to choose the ingredients included in the composition and the amounts thereof so that they do not harm the properties of the compositions of the present invention.

The composition according to the invention is preferably in the form of styling or care gels, care lotions or creams, conditioners, masks or sera.

The composition according to the invention can be obtained by mixing at least 2 compositions, one comprising at least ii) one acid as described previously and the other comprising i) one or more dicarbonyl compounds corresponding to formula (I) and/or derivatives thereof and/or hydrates thereof and/or salts thereof as described previously, one or more alkalinizing agents being present in either of the compositions or both.

A subject of the invention is also a process for straightening keratin fibres, such as the hair, using i) 3 to 15% of one or more dicarbonyl compounds corresponding to formula (I) and/or derivatives thereof and/or hydrates thereof and/or salts thereof as defined previously; and ii) one or more acids different from i) as defined previously; and, iii) one or more alkalinizing agents; with iv) a step of straightening by means of a straightening iron at a temperature of at least 150° C., preferably ranging from 150 to 250° C.

In a first variant, the process for straightening the hair comprises the application to the hair of the composition (composition A) described previously, i.e. comprising ingredients i), ii) and optionally iii), followed iv) by a step of straightening by means of a straightening iron at a temperature of at least 150° C., preferably ranging from 150 to 250° C.

In this first variant, the process of the invention comprises the application of the composition A described previously, followed by a step of straightening keratin fibres, such as the hair, with an iron. Straightening with an iron is known from the prior art. It consists in straightening the keratin fibres, in particular the hair, with flat heating tongs, which are generally metallic. The straightening irons are generally used at a temperature ranging from 150 to 250° C.

In a second variant, the process for straightening the hair comprises the successive application to the hair, and in any order with or without intermediate rinsing, of a composition comprising i) one or more dicarbonyl compounds corresponding to formula (I) and/or derivatives thereof and/or hydrates thereof and/or salts thereof as defined previously, the dicarbonyl compounds corresponding to formula (I), and/or derivatives thereof and/or hydrates thereof and/or salts thereof being present in the composition in an amount ranging from 3 to 15% in weight of the weight of the composition, and of a composition comprising ii) one or more acids different from the compound(s) i) as defined previously, and iii) one or more alkalinizing agents which are optionally present in either of the compositions or both, the application of these compositions being followed by a step of straightening by means of a straightening iron at a temperature of at least 150° C., preferably ranging from 150 to 250° C.

In another variant, the process for straightening keratin fibres, in particular the hair, comprises the successive application to said fibres, and in any order with or without intermediate rinsing,
of a composition (composition B) comprising i) 3 to 15% of one or more dicarbonyl compounds corresponding to formula (I) and/or derivatives thereof and/or hydrates thereof and/or salts thereof and, optionally, iii) one or more alkalinizing agents as described previously;
of a composition (composition C) comprising ii) one or more acids different from i) as defined previously and iii) one or more alkalinizing agents as defined previously,
   the application of these compositions
being followed by a step of straightening by means of a straightening iron at a temperature of at least 150° C., preferably ranging from 150 to 250° C.

In this variant, the composition C will preferably be applied before the composition B.

The process of the invention may comprise other intermediate steps aimed at improving the straightening of the keratin fibres.

According to one particular embodiment, the contact time of the composition A or of the compositions B or C on the keratin fibres, such as the hair, ranges from 10 to 60 minutes, preferably from 20 to 40 minutes. After this or these leave-on time or times, straightening with a brush and with a hairdryer (blow-drying) is performed. The hair is then straightened with a straightening iron at a temperature ranging from 150 to 250° C. and preferably ranging from 210 to 230° C.

In another variant, the process for straightening keratin fibres, such as the hair, comprises the successive application to said fibres, and in any order with or without intermediate rinsing,
of a composition comprising i) one or more dicarbonyl compounds corresponding to formula (I) and/or derivatives thereof and/or hydrates thereof and/or salts thereof as defined previously; the dicarbonyl compounds corresponding to formula (I), and/or derivatives thereof and/or hydrates thereof and/or salts thereof being present in the composition in an amount ranging from 3 to 15% in weight of the weight of the composition
of a composition comprising ii) one or more acids different from the compound(s) i) as defined previously; and
of a composition comprising iii) one or more alkalinizing agents, the application of these compositions being followed by a step of straightening by means of a straightening iron at a temperature of at least 150° C., preferably ranging from 150 to 250° C.

According to one particular mode of the invention, i) is applied to the fibres, then ii) and optionally iii).

According to another particular embodiment, ii) and optionally iii) are applied first, and then i).

The process of the invention may comprise the application of other hair agents as a pretreatment or post-treatment. In particular, it may comprise the application of a conditioning care product as a post-treatment.

According to another embodiment, the process for straightening keratin fibres, such as the hair, comprises a step of washing said fibres and then of drying with a hairdryer before application of the composition A or of the compositions B or C. According to this particular embodiment, the steps described above are next, such as the contact times of the compositions, the straightening with the straightening iron, the application of a conditioning agent and the rinsing, it being possible for all these steps to be carried out independently of one another, it been possible for blow-drying to be inserted between the contact of the composition according to the invention and the straightening with the iron. According to one particular embodiment, the straightening with the straightening iron is performed in several passes on the keratin fibres, such as the hair, in general 8 to 10 passes.

The process of the present invention is preferably performed a step of permanent reshaping at basic pH nor based on a reducing agent. According to one particular embodiment of the invention, the composition comprising ingredients i) and ii) and optionally iii) as defined previously do not comprise a reducing agent.

The examples that follow serve to illustrate the invention without, however, being limiting in nature.

### EXAMPLES

The following compositions were prepared:

| Compositions | 1 | 2 | 3 | 4 | 5 | 6 | 7 | 8 | 9 | 10 | 11 |
|---|---|---|---|---|---|---|---|---|---|---|---|
| Glyoxylic acid i) | 5 g | 5 g | 5 g | 5 g | 5 g | | | | | | |
| Lactic acid ii) | | | 3 g | | | | 3 g | | | | 3 g |
| Phosphoric acid ii) | | | | 5 g | | | | 5 g | | | |
| Benzoic acid ii) | | | | | 2 g | | | | 2 g | | |
| Pyruvic acid i) | | | | | | | | | | 8 g | 8 g |
| Glycolic acid ii) | | 3 g | 5 g | 5 g | | 3 g | | | | | |
| Sodium hydroxide iii) | qs | qs | qs | qs | qs | qs | qs | qs | qs | qs | qs |
| | pH 2.2 | pH 2.2 | pH 2.2 | pH 2.2 | pH 2.2 | pH 2.2 | pH 2.2 | pH 2.2 | pH 2.2 | pH 2.2 | pH 2.2 |
| Water | qs | qs | qs | qs | qs | qs | qs | qs | qs | qs | qs |
| | 100 g | 100 g | 100 g | 100 g | 100 g | 100 g | 100 g | 100 g | 100 g | 100 g | 100 g |

Preparation of composition 1: sodium hydroxide at 10% is added to a solution of 5 g of glyoxylic acid and of 75 g of water, with stirring and at ambient temperature, so as to achieve a pH of 2.2, and then the medium is made up to 100 g by adding water.

Preparation of compositions 2 to 5: respectively 3 g, 3 g, 5 g and 2 g of acid according to the composition table above are added, with stirring and at ambient temperature, to a solution of 5 g of glyoxylic acid and of 45 g of water. 20 g of water are added, the pH is adjusted to 2.2 by adding sodium hydroxide at 10% and then the medium is made up to 100 g by adding water.

Preparation of compositions 6 to 9: 3 g, 3 g, 5 g and 2 g of acid according to the composition table above are diluted in 75 g water, the pH is adjusted to 2.2 by adding sodium hydroxide at 10% and then the medium is made up to 100 g by adding water.

Preparation of composition 10: sodium hydroxide at 10% is added to a solution of 8 g of pyruvic acid and of 75 g of water, with stirring and at ambient temperature, so as to achieve a pH of 2.2, and then the medium is made up to 100 g by adding water.

Preparation of compound 11: 3 g of lactic acid are added, with stirring and at ambient temperature, to a solution of 8 g of pyruvic acid and of 45 g of water. 20 g of water are added, the pH is adjusted to 2.2 by adding sodium hydroxide at 10% and then the medium is made up to 100 g by adding water.

### One-Step Process for Treating Keratin Fibres:

Compositions 1 to 11, optionally shaken before use, are applied to curly hair, which may be natural or dyed, or sensitized by a prior bleaching step, at a rate of 1 g per 2 g of hair. After 15 minutes, the hair is rinsed, dried with a hairdryer (blow-drying) and then straightened by passing over it flat tongs brought to 210° C. It is subsequently shampooed to examine the remanence of the straightening effects and of modification of the mechanical and cosmetic properties of the fibres.

Compositions 2, 3, 4 and 5 make it possible to obtain straightening properties which are superior to those obtained with compositions 1 and 6 to 9. The properties in terms of curl relaxation, protection of the natural or artificial colour, resistance of the fibres to mechanical stresses (pulling, rubbing, twisting), sheen, smooth feel and smooth look are superior with compositions 2, 3, 4 and 5.

Likewise, composition 11 produces performance levels which are greater than those obtained with composition 1 or 10.

### Two-Step Process for Treating Keratin Fibres:

According to a two-step process with rinsing with water, one of compositions 6 to 9 (alternatively 1 or 10) is applied to hair, at a rate of 1 g per 2 g of hair, the compositions are left to act for 15 minutes, the hair is rinsed, sponged and dried, and composition 1 or 10 (alternatively 6 to 9) is applied also at a rate of 1 g per 2 g of hair for 15 minutes. The hair is then dried (blow-drying) and then straightened by passing over it flat tongs brought to 210° C. (10 passes on locks separated into two thicknesses).

According to another two-step process without rinsing, one of compositions 6 to 9 (alternatively 1 or 10) is applied to hair, at a rate of 1 g per 2 g of hair, the compositions are left to act for 15 minutes, and then, without rinsing, composition 1 or 10 (alternatively 6 to 9) is applied at a rate of 1 g per 2 g of hair for 15 minutes. The hair is then dried (blow-drying) and then straightened by passing over it flat tongs brought to 210° C. (10 passes on locks separated into two thicknesses).

As with the one-step treatment, the application of one of composition 1 or 10, combined with the application of compositions 6 to 9 produce performance levels which are greater than those obtained with one of compositions 1 to 10 used alone, or one of compositions 6 to 9 used alone.

### Comparative Example

Compositions A and B were prepared with the following compositions:

| | A (invention) | B (Comparative) |
|---|---|---|
| Compositions | (% wt/wt) | (% wt/wt) |
| GLYOXYLIC ACID | 5% AM | 5% AM |
| CITRIC ACID | 3% AM | 3% AM |
| SODIUM HYDROXIDE | 1% AM | — |
| DEIONIZED WATER | Qs 100 | Qs 100 |

2.7 g hair locks of type I grey hair colored with MAJIROUGE 6.66 (MAJIREL) were washed with a shampoo and blow dried. Then 2.7 g of composition A was applied to one of the hair lock and 2.7 g of composition B was applied to another hair lock. After a leave-on time of 20 minutes on the hair, the locks were blow-dried with a hairdryer (brushing with 15 passes of a brush) and were then straightened with a straightening iron (10 passes). The locks were then washed with a shampoo and let dried naturally (spontaneous).

We measure the color of hair locks by using a spectrocolorimeter Konica Minolta CM 2600d (illuminant D65, angle 10°, specular components included) in the L*a*b* system.

According to this system, L* indicates the lightness of the color of the hair. The chromaticity coordinates are expressed by the parameters a* and b*, a* indicating the axis of red/green shades and b* the axis of yellow/blue shades.

ΔE corresponds to the color difference between untreated colored hair and colored hair treated with composition A or B, according to the following equation:

ΔE=[(L*−L₀*)²+((a*−a₀*)²+(b*−b₀*)²]^{1/2}

where L*, a* b* correspond to the colorimetric values for treated colored hair, and L₀*, a₀* b₀* correspond to the colorimetric values for untreated colored hair.

The more important is the ΔE value, the more important the difference of color between the untreated colored hair lock and straightened colored hair lock hair is.

| | L* | a* | b* | ΔE |
|---|---|---|---|---|
| Hair locks colored with Majirouge | 26.97 | 18.15 | 6.62 | — |
| Hair locks colored with Majirouge and | 41 | 17.37 | 16.7 | 17.29 |
| treated with the straightening process | | | | |
| using composition A (invention) | | | | |
| Hair locks colored with Majirouge and | 47.47 | 14.32 | 21.3 | 25.5 |
| treated with the straightening process | | | | |
| using composition B (comparative) | | | | |

It has been observed that the color difference was less important when using composition A of the invention.

1. Cosmetic composition comprising:
i) one or more dicarbonyl compounds corresponding to formula (I) below, and/or derivatives thereof and/or hydrates thereof and/or salts thereof: 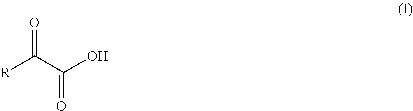
in which formula (I):
R represents an atom or group chosen from i) hydrogen, ii) carboxyl —C(O)—OH, iii) linear or branched C₁-C₆ alkyl which is optionally substituted, preferably with at least one hydroxyl —OH radical, carboxyl radical or halogen radical such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and its tautomers such as 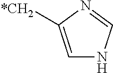
with * representing the part linked to the rest of the molecule; the dicarbonyl compounds corresponding to formula (I), and/or derivatives thereof and/or hydrates thereof and/or salts thereof being present in the composition in an amount ranging from 5 to 15% in weight of the total weight of the composition;
ii) one or more acids different from the compound(s) i) as defined previously; and
iii) one or more alkalinizing agents chosen from i) aqueous ammonia, ii) alkali metal or alkaline-earth metal carbonates or hydrogen carbonates, such as sodium carbonates or hydrogen carbonates or potassium carbonates or hydrogen carbonates, iii) alkali metal or alkaline-earth metal phosphates or (di)hydrogen phosphates, iv) alkali metal or alkaline-earth metal hydroxides, such as sodium or potassium hydroxides, or mixtures thereof, v) alkanolamines, such as monoethanolamine or trihydroxyethylamine, vi) oxyethylenated and/or oxypropylenated ethylenediamines, vii) amino acids and viii) the compounds of formula (II) below: 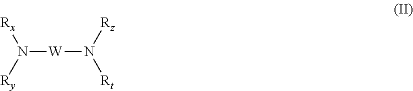
in which formula (II) W is a divalent C₁-C₆ alkylene radical optionally substituted with one or more hydroxyl groups or a C₁-C₆ alkyl radical, and/or optionally interrupted with one or more heteroatoms such as O, or NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

2. Composition according to claim 1, in which the dicarbonyl compound(s) are of formula (I) with R representing i) a hydrogen atom or ii) a linear or branched C₁-C₆ alkyl group optionally substituted with a carboxyl group.

3. Composition according to either one of the preceding claims, in which the dicarbonyl compound(s) of formula (I) and/or derivatives thereof and/or hydrates thereof and/or salts thereof are chosen from glyoxylic acid and pyruvic acid, a derivative thereof, salts thereof and hydrates thereof, preferably from glyoxylic acid, a derivative thereof and the hydrate forms of these compounds.

4. Composition according to any one of the preceding claims, in which the dicarbonyl compound(s) of formula (I) and/or derivatives thereof are chosen from glyoxylic acid esters, glyoxylic acid amides, glyoxylic acid (thio)acetals and hemi(thio)acetals, and glyoxylic acid ester (thio)acetals and hemi(thio)acetals.

5. Composition according to claim 3, in which the glyoxylic acid is in its hydrate form.

6. Composition according to any one of claims 1 to 5, comprising from 5% to 15% by weight of one or more dicarbonyl compounds of formula (I) and/or derivatives thereof and/or hydrates thereof and/or salts thereof, preferably from 5% to 10% by weight of the total weight of the composition.

7. Composition according to any one of the preceding claims, in which the acid(s) ii) is (are) chosen from the following organic or inorganic acids, or mixtures thereof:
hydrochloric acid, sulfuric acid, phosphoric acid;
sulfonic acids Rₐ—S(O)₂—OH, phosphonic acids Rₐ—P(O)(OH)₂, with Rₐ representing an optionally substituted C₁-C₈ alkyl, optionally substituted (hetero)aryl or optionally substituted (hetero)aryl(C₁-C₈)alkyl group;
aromatic or non-aromatic carboxylic acids comprising at least one carboxyl function —C(O)—OH chosen from:
C₂-C₈ monoacids corresponding to the formula R_{b}—C(O)—OH in which the R_{b} radical represents a C₂-C₈ alkyl, (hetero)aryl or (hetero)aryl(C₂-C₈)alkyl group, the alkyl part being linear or branched, the alkyl and/or (hetero)aryl part being optionally substituted, preferably with one or more hydroxyl groups, one of the hydroxyl groups preferably being separated from the carboxyl function —C(O)—OH by one or two carbon atoms;
C₂-C₃₀ diacids corresponding to the formula HO—C(O)—R_{c}—C(O)—OH in which the R_{c} radical represents:
a) a single covalent bond σ;
b) a saturated or unsaturated, acyclic, linear or branched, divalent C₁-C₂₈, in particular C₁-C₁₀, hydrocarbon-based group, which is optionally substituted, preferably with one or more hydroxyl groups, more particularly the divalent hydrocarbon-based group preferably being a C₁-C₈ alkylene group which is optionally substituted with one or more hydroxyl groups or a (C₂-C₆)alkenylene group which is optionally substituted with one or more hydroxyl groups;
c) a (hetero)arylene group which is optionally substituted, preferably with one or more hydroxyl groups and which is preferably an arylene group such as phenylene;
d) a (hetero)cycloalkylene group which is optionally substituted, preferably with one or more hydroxyl groups and which is preferably a cycloalkylene group such as cyclohexylene;
e) or a divalent group resulting from the association of radicals derived from the groups defined in b), c) and/or d) as defined above, such as: -(hetero)aryl(C₁-C₁₀)alkyl-; —(C₁-C₁₀)alkyl(hetero)aryl(C₁-C₁₀)alkyl-; -(hetero)aryl(C₁-C₁₀)alkyl(hetero)aryl-; or -(hetero)cycloalkyl(C₁-C₁₀)alkyl-; and more preferentially -aryl(C₁-C₆)alkyl- such as -phenyl(C₁-C₆)alkyl-;
particularly, the diacids are chosen from those in which R_{c} represents a), b) or c);
polyacids corresponding to the formula R_{d}[C(O)—OH]ₓ with x representing an integer greater than or equal to 3, preferably x ranging from 3 to 6, more particularly from 3 to 4 and in particular such that x is equal to 3; and R_{d} represents a polyvalent group chosen from:
1) a saturated or unsaturated, acyclic, linear or branched, polyvalent C₁-C₂₈, in particular C₂-C₂₀, hydrocarbon-based group, which is optionally substituted with one or more groups, preferably hydroxyl groups, the hydrocarbon-based group preferably being a trivalent C₂-C₈ group which is optionally substituted with one or more hydroxyl groups;
2) a polyvalent (hetero)aryl group which is optionally substituted, preferably with one or more hydroxyl groups, which is preferably an at least trivalent aryl group such as phenyl;
3) a polyvalent (hetero)cycloalkyl group which is optionally substituted, preferably with one or more hydroxyl groups, which is preferably a cycloalkyl group such as cyclohexyl;
4) or a polyvalent group resulting from the association of radicals derived from the groups defined in 1), 2) and/or 3), such as: (hetero)aryl(C₁-C₁₀)alkyl; (C₁-C₁₀)alkyl(hetero)aryl(C₁-C₁₀)alkyl; (hetero)aryl(C₁-C₁₀)alkyl(hetero)aryl; or (hetero)cycloalkyl(C₁-C₁₀)alkyl; and more preferentially aryl(C₁-C₆)alkyl such as phenyl(C₁-C₆)alkyl;
more particularly, the polyacids being chosen from the triacids derived from groups defined in 1), in particular of C₅-C₂₀;
aromatic or non-aromatic sulfocarboxylic acids comprising at least one carboxyl function —C(O)—OH and at least one sulfonic function —S(O)₂—OH, such as [HO—C(O)]_{y}—R_{d}—[S(O)₂—OH]_{z} with R_{d} as defined previously for the polyacids; y and z being integers greater than or equal to 1, the sum y+z preferably being greater than or equal to 2 such as equal to 3; the sulfocarboxylic acids preferably being of C₂-C₁₀, and the sulfonic acid group being separated from the carboxylic acid group(s) by a polyvalent (C₁-C₆)alkyl or aryl(C₁-C₆)alkyl chain, the alkyl part of which is linear or branched, optionally substituted with a hydroxyl group;
aromatic or non-aromatic phosphocarboxylic acids comprising at least one carboxyl function —C(O)—OH and at least one phosphonic function —P(O)(OH)₂, such as [HO—C(O)]_{y}—R_{d}—[P(O)(OH)₂]Z with R_{d} as defined previously for the polyacids; y and z being integers greater than or equal to 1, the sum y+z preferably being greater than or equal to 2 such as equal to 3; the phosphocarboxylic acids in particular being of C₂-C₁₀, and the phosphonic acid group being separated from the carboxylic acid group(s) by a polyvalent (C₁-C₆)alkyl or aryl(C₁-C₆)alkyl chain, the alkyl part of which is linear or branched and optionally substituted with a hydroxyl group.

8. Composition according to any one of the preceding claims, in which the acid(s) ii) is (are) chosen from organic acids.

9. Composition according to any one of the preceding claims, in which the acid(s) ii) is (are) chosen from aromatic or non-aromatic carboxylic acids comprising at least one carboxyl function —C(O)—OH.

10. Composition according to any one of the preceding claims, in which the acid(s) ii) is (are) chosen from the monocarboxylic acids as defined in claim 7.

11. Composition according to any one of the preceding claims, in which the acid(s) ii) is (are) chosen from inorganic acids.

12. Composition according to any one of the preceding claims, in which the acid(s) ii) is (are) chosen from phosphoric acid, glycolic acid, lactic acid, benzoic acid and salicylic acid; oxalic acid, malonic acid, hydroxymalonic acid, succinic acid, malic acid, tartaric acid, maleic acid, fumaric acid, itaconic acid, glutaric acid, adipic acid, azelaic acid and sebacic acid, phthalic acid, isophthalic acid and terephthalic acid; sulfosuccinic acid, para-sulfobenzoic acid, 4-sulfosalicylic acid, phosphoglycolic acid and citric acid; preferably, the acid(s) ii) is (are) chosen from glycolic acid, lactic acid and benzoic acid.

13. Composition according to any one of the preceding claims, which comprises a minimal content of acids ii) as defined in any one of claims 1 and 7 to 12 greater than or equal to 1% by weight, preferably greater than or equal to 2% by weight, the content of acid(s) ii) which is (are) different from the dicarbonyl derivatives of formula (I) as defined in any one of claims 1 to 5 ranging even more preferentially from 2% to 10% by weight relative to the total weight of the composition.

14. Composition according to any one of the preceding claims, which has a pH greater than or equal to 1.7 and less than 7, particularly ranging from 1 to 4, more particularly ranging from 1 to 3, and preferentially ranging from 1.7 to 3.

15. Composition according to any one of the preceding claims, characterized in that it is aqueous and comprises water at a concentration particularly ranging from 5% to 98%, more particularly ranging from 5% to 90%, and preferentially ranging from 10% to 90% by weight relative to the total weight of the composition.

16. Process for straightening keratin fibres, such as the hair, using i) one or more dicarbonyl compounds of formula (I) and/or derivatives thereof and/or hydrates thereof and/or salts thereof as described in any one of claims 1 to 6; ii) one or more acids as described in any one of claims 1 and 7 to 13; iii) one or more alkalinizing agents as described in claim 1; the amount of dicarbonyl compounds of formula (I) and/or derivatives thereof and/or hydrates thereof and/or salts in a composition containing them ranging from 3 to 15% in weight of the total weight of the composition, with iv) a step of straightening by means of a straightening iron at a temperature of at least 150° C., preferably ranging from 150 to 250° C.

17. Process for straightening keratin fibres according to the preceding claim, which comprises the application to said fibres of the composition according to any one of claims 1 to 16, followed by a contact time of between 10 and 60 minutes, followed by a step of straightening by means of a straightening iron at a temperature of at least 150° C., preferably ranging from 150 to 250° C.

18. Process for straightening keratin fibres according to claim 16, which comprises the successive application to said fibres, and in any order with or without intermediate rinsing, of a composition comprising i) one or more dicarbonyl compounds corresponding to formula (I) and/or derivatives thereof and/or hydrates thereof and/or salts thereof as described in any one of claims 1 to 6, the dicarbonyl compounds corresponding to formula (I), and/or derivatives thereof and/or hydrates thereof and/or salts thereof being present in the composition in an amount ranging from 3 to 15% in weight of the weight of the composition; of a composition comprising ii) one or more acids as defined in any one of claims 1 and 7 to 13; and iii) one or more alkalinizing agents, preferably as described in claim 1, said alkalinizing agents being present in either of the compositions or both, or in a supplementary composition;
the application of these compositions being followed by a contact time for each of them with said fibres of from 10 to 60 minutes and then by a step of straightening said fibres by means of a straightening iron at a temperature of at least 150° C., preferably ranging from 150 to 250° C.

19. Use of a composition comprising i) one or more dicarbonyl compounds corresponding to formula (I) below, and/or derivatives thereof and/or hydrates thereof and/or salts thereof: 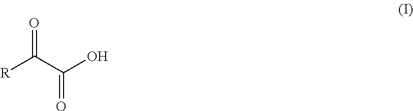
in which formula (I):
R represents an atom or group chosen from i) hydrogen, ii) carboxyl —C(O)—OH, iii) linear or branched C₁-C₆ alkyl which is optionally substituted, preferably with at least one hydroxyl —OH radical, carboxyl radical or halogen radical such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and its tautomers such as 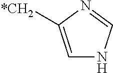
with * representing the part linked to the rest of the molecule; the dicarbonyl compounds corresponding to formula (I), and/or derivatives thereof and/or hydrates thereof and/or salts thereof being present in the composition in an amount ranging from 3 to 15% in weight of the total weight of the composition;
ii) one or more acids different from the compound(s) i) as defined previously; and
iii) one or more alkalinizing agents, for straightening/relaxing keratin fibres, in particular the hair.
